(19) United States
(12) Patent Application Publication
(10) Pub. No.: US 2022/0204554 A1
(43) Pub. Date: Jun. 30, 2022

(54) METHOD FOR METAL-FREE PURIFICATION OF PROTEIN FROM A PROTEIN MIXTURE OR A CELL LYSATE WITH THE N-TERMINUS GLYCINE TAGGING

(71) Applicant: INDIAN INSTITUTE OF SCIENCE EDUCATION AND RESEARCH BHOPAL, Bhauri, Bhopal, Madhya Pradesh (IN)

(72) Inventor: Vishal RAI, Bhauri (IN)

(73) Assignee: INDIAN INSTITUTE OF SCIENCE EDUCATION AND RESEARCH BHOPAL, Bhauri, Bhopal, Madhya Pradesh (IN)

(21) Appl. No.: 17/605,579

(22) PCT Filed: Apr. 17, 2020
(86) PCT No.: PCT/IN2020/050363

§ 371 Date: Oct. 22, 2021

(30) Foreign Application Priority Data
Apr. 22, 2019 (IN) 201921015806

(51) Int. Cl. C07K 1/22

(52) U.S. Cl. CPC C07K 1/22

(57) ABSTRACT

The invention pertains to the method of N-terminus Glycine tagged metal-free protein purification by selective labeling of N-terminus Gly containing proteins, its capture, and release through modified resin under mild operating conditions. The selective labeling of N-terminus Glycine enables the formation of an aminoalcohol. The invention is for selective tagging of N-Gly in a protein. The invention is for separation of immobilised N-terminus glycine proteins from the functionalised resin under mild aqueous physiological conditions by C—C bond dissociation with additive, in which the additive enables the resonance-assisted electron density (RED) polarization to facilitate C—C bond dissociation. The invention provides the N-Gly specific installation of a probe in a protein within cell lysate. The invention covers the special aldehydes, including its on-resin derivative, for the given purpose.

### FIELD OF INVENTION

The invention pertains to protein chemistry with special reference to protein labelling and metal-free protein purification.

### BACKGROUND

Living systems are intricate, and a broad set of proteins drive their complex machinery. Elucidating the biological role of these individual proteins requires investigation of their physical, chemical, and structural properties. In turn, it necessitates the production of a pure functional protein of interest (POI). The recombinant protein expression caters to the growing academic and industrial demands for the synthesis of proteins. The other end of the spectrum of the synthesis of proteins is the purification of the synthesized protein of interest with retaining the functionality and is still a challenge. Purification of the synthesized proteins requires the isolation of POI from the cell extract. The fishing out of a particular protein among thousands of proteins rendering similar features, is highly demanding. It needs a methodology to synchronize diverse aspects of chemical reactivity and selectivity. Besides, the restriction to operate under mild physiological conditions amplifies the complexity.

Analytically pure proteins are indispensable for studies on their structure, post-translational modifications, and function. Affinity tag-based approach is the widely accepted method for their purification. The initial efforts involved the development of affinity tags that can render unique capture and release attributes. In this perspective, immobilized metal-affinity chromatography is one of the most prominent techniques. A sequence of His residues (His tag) installed in a protein provides for the preferential binding to a metal complex, and limitation of such method was the non-specific binding to other residues in the proteins and leaching of metal. It motivated research on metal-free techniques and specific non-covalent interactions, which led to the development of peptide and protein-based fusion tags that operate under mild conditions. The specificity in these cases requires a large recognition motif, either as a part of the protein or as the capture ligand on a resin. Even in this method, the loss of protein is unavoidable due to the participation of multiple dynamic interactions that provide a gradient of binding energy. It has been the prime reason behind the lack of methods for covalent affinity chromatography. The problem has been addressed to some extent indirectly by coding an additional enzyme-cleavable fragment, e.g., Halo-Tag. Here, a protein with the tag is installed on resin and allows stringent washing with minimal loss of the POI. Protein purification by this method still was a limitation and challenge as the removal of the tag was essential due to its size (˜34 kDa), making its removal an essential step, and protease releases the POI leaving behind the Halo-Tag on the resin. Unfortunately, the resin is not recyclable, and the separation of protease from POI requires an additional step. Earlier, we developed methods for the single-site labelling of POI, which offered a discrete switch-on mechanism for its capture through late-stage covalent immobilization. However, translation of the chemical transformation to enable the capture process to the solid-phase, i.e., immobilisation of the single amino acid tagged POI onto a functionalized solid-phase matrix, and the development of a method for release of said POIs in physiological conditions had posed monumental challenges. The major challenge for translation of a method to covalent affinity chromatography remained a problem to be solved and required solving the puzzle of protein release under mild conditions. Thus, the present invention addresses the problems of protein purification adopting single-site labelling, which offers a discrete switch-on mechanism for its capture, and a method for release in near-physiological conditions.

### OBJECT OF INVENTION

An object of the invention is for method of metal-free purification of protein comprising of functionalized resin with N-terminus glycine capture reagent, immobilisation, and separation of the N-terminus glycine protein from a protein mixture or cell lysate under mild aqueous physiological conditions.

Another object of the invention is for method for metal-free purification of protein from a protein mixture or cell lysate comprising reacting the N-terminus glycine capture reagent with N-terminus glycine containing proteins in an aqueous phase from the protein mixture or cell lysate to form N-terminus glycine tagged protein and reacting N-terminus glycine tagged proteins with the resin or a probe to form a C—C bond association and stable amino alcohol; and separation of the N-terminus glycine protein from a protein mixture or cell lysate from the resin or probe under mild aqueous physiological conditions.

Another object of the invention is for immobilising the N-terminus glycine containing proteins in an ordered pattern from the protein mixture or cell lysate on the functionalized resin.

Yet another object of the invention is for separation of immobilised N-terminus glycine proteins from the functionalised resin under mild aqueous physiological conditions by C—C bond dissociation with additive, in which the additive enables the resonance-assisted electron density (RED) polarization to facilitate C—C bond dissociation.

Another embodiment of the invention is for the recovery and recycling of the functionalized resin without substantial loss of activity.

### BRIEF DESCRIPTION OF THE DRAWINGS AND FIGURES

FIG. 1 depicts the common methods for isolation of a protein under physiological conditions. It includes affinity chromatography under mild conditions (x-y) enabled by small tag (x-z) or metal-free non-covalent interactions (y-z).

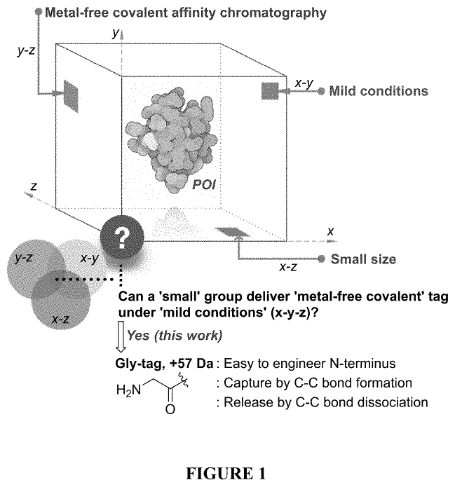

FIG. 2 depicts a) Selective labeling of N-terminus glycine containing proteins with the capture reagents. b) N-terminus Glycine-tag enabled protein purification: protein capture (step 1) through modified sepharose resin and its release (step 2) under mild operating conditions.

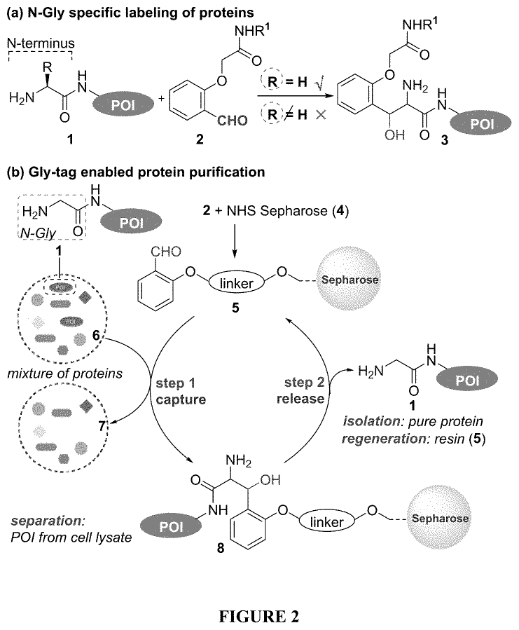

FIG. 3 depicts Single-site N-terminus Glycine labeling of several proteins.

FIG. 4 depicts N-terminus glycine labelled proteins with capture reagents further tagging with probes and isolation of analytically pure tagged proteins.

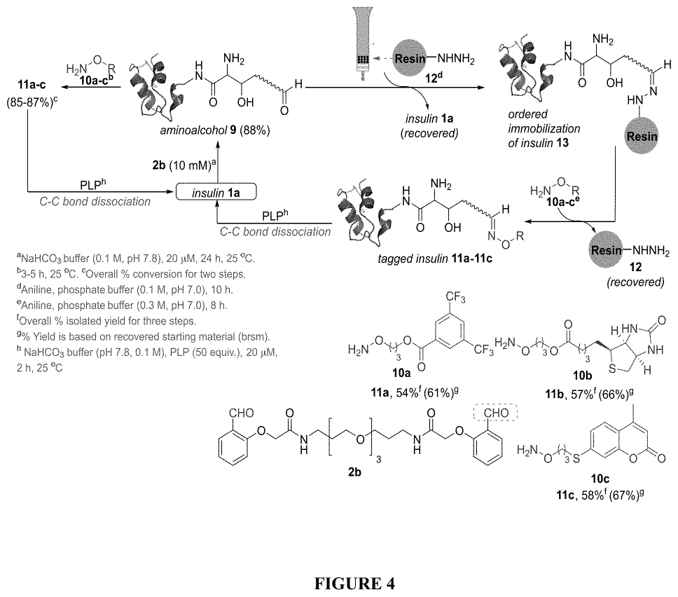

FIG. 5 depicts recombinantly expressed protein with its N-Glycine specific labeling in the cell lysate.

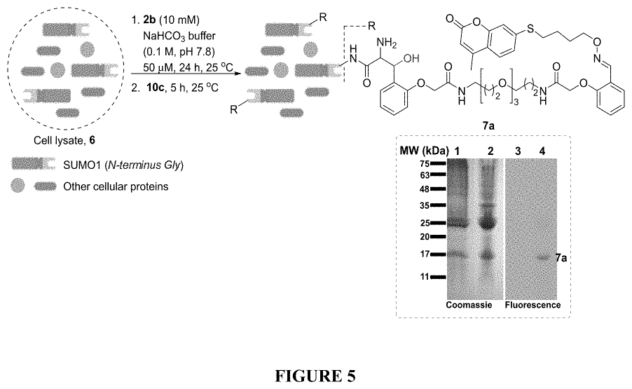

FIG. 6 (a) HPLC spectrum of 2c. (b) ESI-MS spectrum of 2c. (2c is N-(3-(2-(2-(3-aminopropoxy)ethoxy)ethoxy)propyl)-2-(2-formylphenoxy) acetamide.

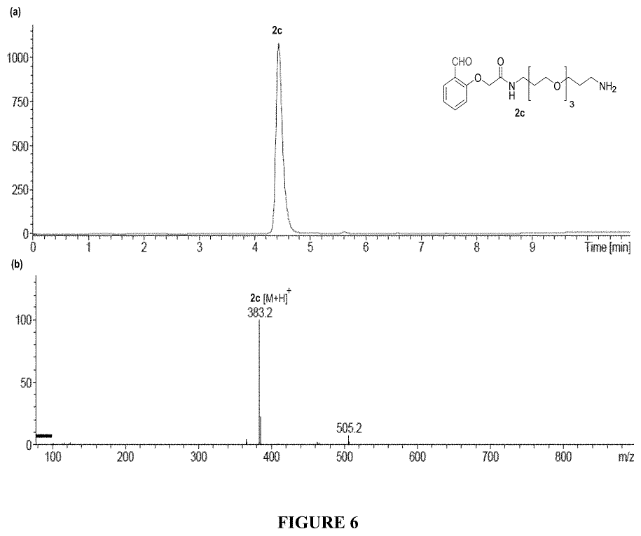

FIG. 7 (a) Immobilization of reagent (2c) on NHS sepharose resin; (b) UV spectra of reagent 2c at different concentrations; (c) Determination of molar extinction coefficient for the reagent 2c; (d) UV spectra of the eluted fraction containing unbound reagent after 2 h; (e) UV spectra of first wash fraction which has the adsorbed reagent.

FIG. 8 depicts the N-terminus labelling of glycine of protein with the glycine capture reagent 2b-N,N-(((oxybis(ethane-2,1-diyl))bis(oxy))bis(propane-3,1-diyl))bis(2-(2-formylphenoxy)acetamide) and ESI-MS spectra of the labelled N-terminus Glycine containing protein.

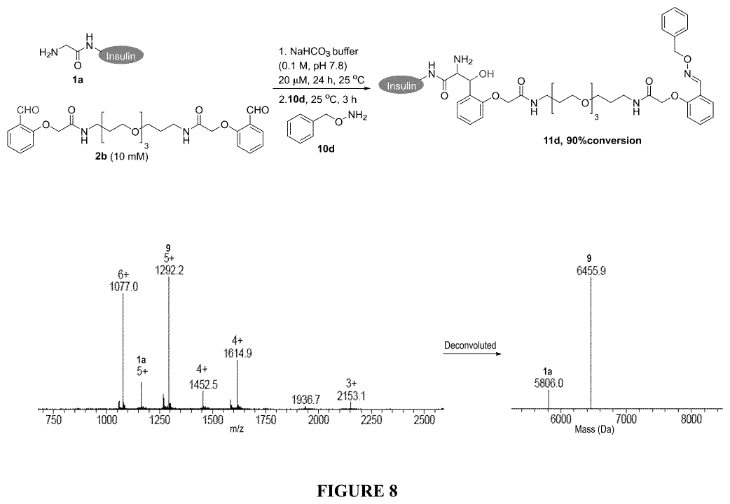

FIG. 9 showing the (a) capture of insulin; UV spectrum of (b) Insulin, (c) Myoglobin, and (d) RNase A, before and after immobilization on the functionalized sepharose resin.

FIG. 10 showing C—C bond dissociation by resonance-assisted electron density (RED) polarization with additives under aqueous physiological conditions. a) Screening of reagents for C—C bond dissociation in aminoalcohol. b) Mechanism of the reaction.

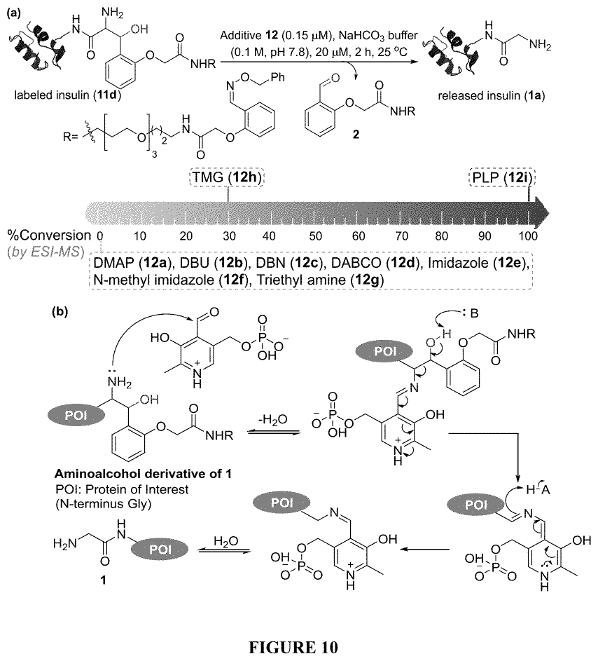

FIG. 11 showing a) Release of immobilized proteins by C—C bond dissociation by resonance-assisted electron density (RED) polarization with additives. b) ESI-MS spectrum of released insulin 1a. c) ESI-MS spectrum of released myoglobin 1b.

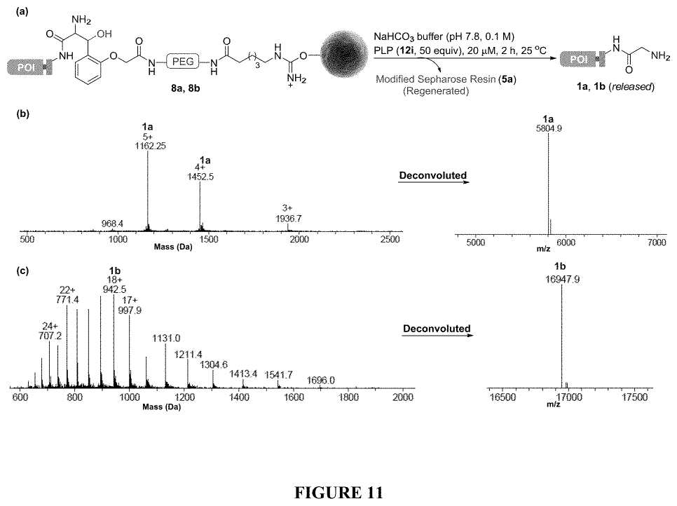

### DESCRIPTION OF THE INVENTION

The invention is described in detail in the description below are provided as an illustration and are not intended to restrict scope of invention in any manner. Any embodiments that may be apparent to a person skilled in the art are deemed to fall within the scope of the present invention.

Accordingly, the invention is for a method of metal-free purification of protein comprising of functionalized resin with N-terminus glycine capture reagent, immobilisation and separation of the N-terminus glycine protein from a protein mixture or cell lysate under mild aqueous physiological conditions.

In an aspect the invention is for a method for metal-free purification of protein from a protein mixture or cell lysate comprising reacting the N-terminus glycine capture reagent with N-terminus glycine containing proteins in an aqueous phase from the protein mixture or cell lysate to form N-terminus glycine tagged protein and reacting N-terminus glycine tagged proteins with the resin or a probe to form a C—C bond association and stable amino alcohol; and separation of the N-terminus glycine containing protein from a protein mixture or cell lysate from the resin or probe under mild aqueous physiological conditions.

In one embodiment, the invention discloses the activation of the N-terminus Glycine in proteins with the glycine capture reagent for the formation of stable aminoalcohol. It enables the labelling of N-terminus Glycine in proteins with remarkable efficiency and selectivity for covalent, selective, and reversible immobilization on the resin.

In another embodiment, the invention discloses a functionalized sepharose resin synthesised with the glycine capture reagents to capture the N-terminus Glycine containing protein selectively, leaving the other proteins in solution.

In another embodiment, the invention discloses a method for the release of immobilised N-terminus Glycine containing protein along with the recovery of functionalized sepharose resin under mild conditions.

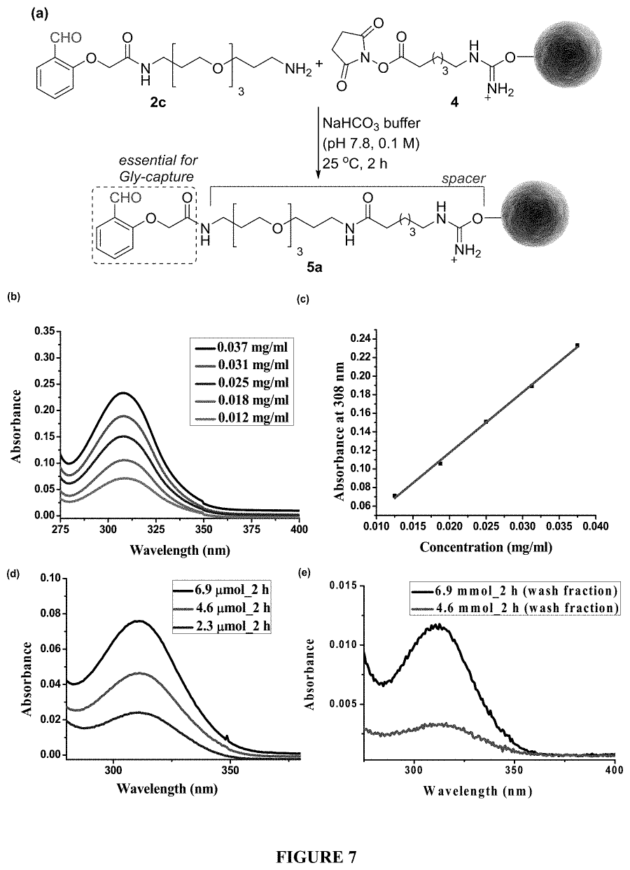

An aspect discloses the synthesis of the glycine capture reagents. Firstly, the aldehyde with proximal hydrogen bond acceptors (2c, FIG. 7) was synthesised in four steps. The N-terminus Glycine capture element was tethered to a PEG diamine and placed a nucleophilic amine functionality at the other end of the reagent (2c). PEG linker regulates the surface availability of the reagent upon its immobilization. On the other hand, amine functionality is provided by the nucleophilic handle to conjugate it with the electrophilic NHS ester functionalized resin. The extent of immobilization of the Glycine capture reagent on to the sepharose resin was quantified by UV analysis (FIG. 7). Initially, the concentration of the NHS was monitored at 260 nm (λₘₐₓ). The reagent 2c also contributes to the absorption at this wavelength, an additional absorbance of the unbound reagent 2c at 308 nm was measured to monitor the extent of immobilization. A standard calibration curve from 2c at different concentrations of the reagent was derived (FIG. 7). Subsequently, we determined the molar extinction coefficient of the reagent 2c. It is further disclosed that two equivalents of reagent 2c was enough to result in 20 μmol/mL of loading.

In one embodiment, therapeutic protein insulin (1a) was examined using the method for immobilization of N-terminus Glycine containing proteins with functionalized resin (5a). Insulin (1a) has two chains where N^{α}—NH₂ of chain B is Phe, and that of chain A is a Gly residue. The N-terminus Glycine formed a stable aminoalcohol with the functionalized resin 5a with the glycine capture reagent 2b, thereby immobilizing the insulin. Further, the invention discloses indicating excellent binding (>90% efficiency). The robust immobilization through C—C bond formation renders ordered single-site immobilization and opens a gateway for protein purification.

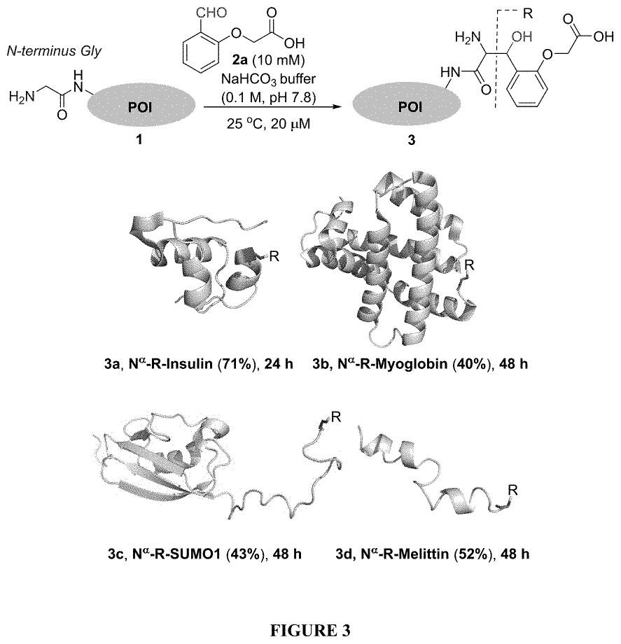

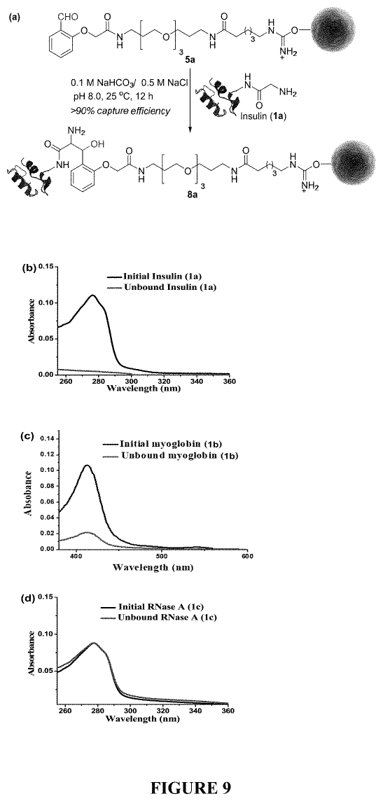

In another aspect several proteins for single-site N-terminus Glycine labeling was carried and the percent of labelling was 71% within 24 h for insulin (FIG. 3, 3a), 40% within 48 h for myoglobin (FIG. 3, 3b), 43% within 48 h for SUMO 1 recombinant protein (FIGS. 3, 3c), and 52% within 48 h for melittin (FIG. 3, 3d), and the % binding was zero for RNAase A (FIG. 9) as RNAase A lacks N-terminus glycine.

In an aspect, the invention discloses a method for metal-free purification of protein from a protein mixture or cell lysate comprising the steps of:
preparing the N-terminus glycine capture reagent;
preparing a functionalized resin with N-terminus glycine capture reagent;
reacting the N-terminus glycine containing proteins from the protein mixture or cell lysate with the functionalized resin to form a C—C bond association and stable amino alcohol;
immobilising the N-terminus glycine containing proteins in an ordered pattern from the protein mixture or cell lysate on the functionalized resin;
separating the N-terminus glycine proteins from the functionalised resin under aqueous physiological conditions by C—C bond dissociation with additive, wherein the additive enables the resonance-assisted electron density (RED) polarization to facilitate C—C bond dissociation; and recovery and recycling of the functionalized resin without substantial loss of activity.

In one embodiment the invention discloses the N-terminus glycine capture reagent selected from the compounds of formula

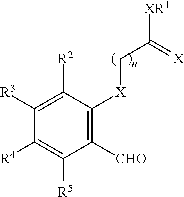

where, X is a heteroatom (O, N, S), n is 1-6 and R¹-R⁵ are independently selected H; alkyl; lower alkyl; cycloalkyl; aryl; heteroaryl; alkenyl; heterocycle; halides; nitro; —C(O)OR* wherein R* is selected from H, alkyl; cycloalkyl and aryl; —C(O)NR**R***, wherein R** and R*** are independently selected from H, alkyl; cycloalkyl and aryl; —CH₂C(O)Rₐ, wherein Rₐ is selected from —OH, lower alkyl, cycloalkyl; aryl, -lower alkyl-aryl, -cycloalkyl-aryl; or —NR_{b}R_{c}, where R_{b} and R_{c} are independently selected from H, lower alkyl, cycloalkyl; aryl or -lower alkyl-aryl; —C(O)R_{d}, wherein R_{d} is selected from lower alkyl, cycloalkyl; aryl or -lower alkyl-aryl; or -lower alkyl-ORₑ, wherein Rₑ is a suitable protecting group or OH group. R¹ group can also be selected from an amino acid, small peptide, large peptide, a protein, an antibody, their unnatural derivatives or other biomolecules bearing —CH₂NH₂ group. Small peptide is a 2-mer to 10-mer peptide and large peptide is 11-mer to 30-mer peptide. All the Rⁿ groups are optionally substituted at one or more substitutable positions with one or more suitable substituents;
the “suitable substituent” includes independently H; hydroxyl; cyano; alkyl, such as lower alkyl, such as methyl, ethyl, propyl, n-butyl, t-butyl, hexyl and the like; alkoxy, such as lower alkoxy such as methoxy, ethoxy, and the like; aryloxy, such as phenoxy and the like; vinyl; alkenyl, such as hexenyl and the like; alkynyl; formyl; haloalkyl, such as lower haloalkyl which includes CF₃, CCl₃ and the like; halide; aryl, such as phenyl and napthyl; heteroaryl, such as thienyl and furanyl and the like; amide such as C(O)NR**R***, where R** and R*** are independently selected from lower alkyl, aryl or benzyl, and the like; acyl, such as C(O)—C₆H₅, and the like; ester such as —C(O)OCH₃ the like; ethers and thioethers, such as O—Bn and the like; thioalkoxy; phosphino; and —NR_{b}R_{c}, where R_{b} and R_{c} are independently selected from lower alkyl, aryl or benzyl, and the like. The term “lower alkyl” as used herein either alone or in combination with another substituent means acyclic, straight or branched chain alkyl substituent containing from one to six carbons and includes for example, methyl, ethyl, 1-methylethyl, 1-methylpropyl, 2-methylpropyl, and the like. A similar use of the term is to be understood for “lower alkoxy”, “lower thioalkyl”, “lower alkenyl” and the like in respect of the number of carbon atoms. For example, “lower alkoxy” as used herein includes methoxy, ethoxy, t-butoxy;
the term “alkyl” encompasses lower alkyl, and also includes alkyl groups having more than six carbon atoms, such as, for example, acyclic, straight or branched chain alkyl substituents having seven to ten carbon atoms;
the term “aryl” as used herein, either alone or in combination with another substituent, means an aromatic monocyclic system or an aromatic polycyclic system. For example, the term “aryl” includes a phenyl or a napthyl ring, and may also include larger aromatic polycyclic systems, such as fluorescent (eg. anthracene) or radioactive labels and their derivatives;
the term “heteroaryl” as used herein, either alone or in combination with another substituent means a 5, 6, or 7-membered unsaturated heterocycle containing from one to 4 heteroatoms selected from nitrogen, oxygen, and sulphur and which form an aromatic system. The term “heteroaryl” also includes a polycyclic aromatic system comprising a 5, 6, or 7-membered unsaturated heterocycle containing from one to 4 heteroatoms selected from nitrogen, oxygen, and sulphur;
the term “cycloalkyl” as used herein, either alone or in combination with another substituent, means a cycloalkyl substituent that includes for example, but is not limited to, cyclopropyl, cyclobutyl, cyclopentyl, cyclohexyl and cycloheptyl. The term also involves “cycloalkyl-alkyl” that means an alkyl radical to which a cycloalkyl radical is directly linked; and includes, but is not limited to, cyclopropylmethyl, cyclobutylmethyl, cyclopentylmethyl, 1-cyclopentylethyl, 2-cyclopentylethyl, cyclohexylmethyl, 1-cyclohexylethyl and 2-cyclohexylethyl. A similar use of the “alkyl” or “lower alkyl” terms is to be understood for aryl-alkyl-, aryl-lower alkyl- (eg. benzyl), -lower alkyl-alkenyl (eg. allyl), heteroaryl-alkyl-, and the like as used herein. For example, the term “aryl-alkyl-” means an alkyl radical, to which an aryl is bonded. Examples of aryl-alkyl- include, but are not limited to, benzyl (phenylmethyl), 1-phenylethyl, 2-phenylethyl and phenylpropyl. As used herein, the term “heterocycle”, either alone or in combination with another radical, means a monovalent radical derived by removal of a hydrogen from a three- to seven-membered saturated or unsaturated (including aromatic) heterocycle containing from one to four heteroatoms selected from nitrogen, oxygen and sulfur. Examples of such heterocycles include, but are not limited to, pyrrolidine, tetra-hydrofuran, thiazolidine, pyrrole, thiophene, hydantoin, diazepine, imidazole, isoxazole, thiazole, tetrazole, piperidine, piperazine, homopiperidine, homo-piperazine, 1,4-dioxane, 4-morpholine, 4-thiomorpholine, pyridine, pyridine-N-oxide or pyrimidine, and the like;
the term “alkenyl”, as used herein, either alone or in combination with another radical, is intended to mean an unsaturated, acyclic straight chain radical containing two or more carbon atoms, at least two of which are bonded to each other by a double bond. Examples of such radicals include, but are not limited to, ethenyl (vinyl), 1-propenyl, 2-propenyl, and 1-butenyl. The term “alkynyl”, as used herein is intended to mean an unsaturated, acyclic straight chain radical containing two or more carbon atoms, at least two of which are bonded to each other by a triple bond. Examples of such radicals include, but are not limited to, ethynyl, 1-propynyl, 2-propynyl, and 1-butynyl;
the term “alkoxy” as used herein, either alone or in combination with another radical, means the radical —O—(C₁₋ₓ) alkyl wherein alkyl is as defined above containing 1 or more carbon atoms, and includes for example methoxy, ethoxy, propoxy, 1-methylethoxy, butoxy and 1,1-dimethylethoxy. Where x is 1 to 6, the term “lower alkoxy” applies, as noted above, whereas the term “alkoxy” encompasses “lower alkoxy” as well as alkoxy groups where x is greater than 6 (for example, x=7 to 10). The term “aryloxy” as used herein alone or in combination with another radical means —O-aryl, wherein aryl is defined as noted above.

In an aspect, the N-terminus glycine capture reagent is preferably N-(3-(2-(2-(3-aminopropoxy)ethoxy)ethoxy)propyl)-2-(2-formylphenoxy)acetamide.

In another aspect, the method discloses a mild aqueous physiological condition at pH of 7±1.

In another aspect, the resin for functionalisation is selected from one of NHS Sepharose, NHS Agarose, and the like.

In one embodiment the additive for C—C bond dissociation is selected from one of 4-dimethyl amino pyridine (DMAP), 1,8-Diazabicyclo[5.4.0]undec-7-ene (DBU), 1,5-Diazabicyclo[4.3.0]non-5-ene (DBN), 1,4-Diazabicyclo[2.2.2]octane (DABCO), Imidazole, N-methyl Imidazole, triethyl amine, pyridoxal-5-phosphate (PLP), or other RED polarization promoting additives and is preferably pyridoxal-5-phosphate.

The invention further discloses that the recovered functionalized resin is used for 5-7 purification cycles.

In another embodiment, the invention is for a method for metal free purification of protein from a protein mixture or cell lysate comprising the steps of:
preparing an N-terminus glycine capture reagent;
reacting the N-terminus glycine capture reagent with N-terminus glycine containing proteins in an aqueous phase from the protein mixture or cell lysate to form N-terminus glycine tagged protein;
reacting N-terminus glycine tagged proteins with the resin or a probe to form a C—C bond association and a stable amino alcohol;
separating the N-terminus glycine proteins from the resin or a probe under aqueous physiological conditions by C—C bond dissociation with additive, wherein the additive enables the resonance-assisted electron density (RED) polarization to facilitate C—C bond dissociation; and optionally recovering and recycling as functionalised resin or a probe without substantial loss of activity.

In an aspect, the N-terminus glycine capture reagent is preferably N,N′-(((oxybis(ethane-2,1-diyl))bis(oxy))bis(propane-3,1-diyl))bis(2-(2formylphenoxy)acetamide).

In another aspect, the method discloses a mild aqueous physiological condition at pH of 7±1.

In another aspect, the resin for functionalisation is selected from one of NHS Sepharose, NHS Agarose, and the like.

In one embodiment, the additive for C—C bond dissociation is selected from one of 4-dimethyl amino pyridine (DMAP), 1,8-Diazabicyclo[5.4.0]undec-7-ene (DBU), 1,5-Diazabicyclo[4.3.0]non-5-ene (DBN), 1,4-Diazabicyclo[2.2.2]octane (DABCO), Imidazole, N-methyl Imidazole, triethyl amine, pyridoxal-5-phosphate (PLP), or other RED polarization promoting additives and is preferably pyridoxal-5-phosphate.

The invention further discloses that the recovered functionalized resin is used for 5-7 purification cycles.

### Examples

The invention is described in detail in the above figures and description, and the following examples below are provided as an illustration and are not intended to restrict the scope of the invention in any manner. Any embodiments that may be apparent to a person skilled in the art are deemed to fall within the scope of the present invention.

### Materials and General Information

The reagents, proteins, and enzymes were purchased from Sigma-Aldrich, Alfa Aeser and Merck Novabiochem. Hydrazide agarose beads were purchased from Thermo Scientific. Boronic acid (polymer bound) was purchased from Sigma Aldrich. The organic solvents used were reagent grade. Aqueous buffers were prepared freshly using Millipore Grade I water (Resistivity>5 MΩ cm, Conductivity<0.2 μS/cm, TOC<30 ppb). Mettler Toledo (FE20) pH meter was used to adjust the final pH. The reaction mixture for the small molecules was stirred (Heidolph, 500-600 rpm). Proteins were either vortexed or incubated in incubator-shaker Thermo Scientific MaxQ 8000 (350 rpm, 25-37° C.). Amicon® Ultra-0.5 mL 3-kDa or 10-kDa MWCO Centrifugal Filters from Merck Millipore was used to remove small molecules from protein mixture, desalting and buffer exchange. Organic solvents were removed by BUCHI rotavapor R-210/215 whereas aqueous samples were lyophilized by CHRiST ALPHA 2-4 LD plus lyophilizer. Circular Dichroism (CD) measurements were recorded on JASCO J-815 CD spectropolarimeter equipped with Peltier temperature controller. All the spectra were measured with a scan speed of 50 nm/min, spectral band width 1 nm using 1 mm path length cuvette at 25° C. UV-Vis spectra was recorded in Agilent Carry-100 UV-Vis Spectrophotometer connected with peltier temperature controller.

### Chromatography

Thin-layer chromatography (TLC) was performed on silica gel coated aluminium TLC plates (Merck, TLC Silica gel 60 F254). The compounds were visualized using a UV lamp (254 nm) and stains such as iodine, ninhydrin, 2,4-diphenylhydrazine. The flash column chromatography of reagents was carried out on Combiflash Rf 200 or gravity columns using 230-400 or 100-200 mesh silica gel from Merck.

### Nuclear Magnetic Resonance Spectra

¹H, ¹³C and spectra were recorded on Bruker Avance III 400 and 500 MHz NMR spectrometer. ¹H NMR spectra were referenced to TMS (0 ppm) CDCl₃ (7.26 ppm), whereas ¹³C NMR spectra were referenced to CDCl₃ (77.16 ppm). Peak multiplicities are designated by the following abbreviations: s, singlet; bs, broad singlet; d, doublet; t, triplet; q, quartet; m, multiplet; dd, doublet of doublets; ddd, doublet of doublet of doublets. Spectra were recorded at 298 K.

### Mass Spectrometry

Agilent Technologies 1200 series HPLC paired to Agilent 6130 mass spectrometer (ESI/APCI) was used for ESI-MS data. HRMS data were recorded on Bruker Daltonics MicroTOF-Q-II with electron spray ionization (ESI). Matrix assisted laser desorption/ionisation time of flight mass spectrometry was performed with Bruker Daltonics UltrafleXtreme Software-Flex control version 3.4, using sinapic acid and α-cyano-4-hydroxycinnamic acid (HCCA) matrix. Data analysis was performed using flex analysis.

### Example 1: Synthesis of N-Terminus Glycine Capture Reagents and Characterization

### A). Synthesis of tert-butyl (3-(2-(2-(3-aminopropoxy)ethoxy)ethoxy)propyl)carbamate (S2)

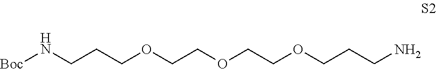 [SMILES: CC(C)(C)OC(=O)NCCCOCCOCCOCCCN]

A solution of 4,7,10-trioxa-1,13-tridecanediamine S1 (34.1 mmol, 7.50 g) in 250 ml round bottom flask and dissolved in DCM (100 mL) followed by slow addition of Boc anhydride (16.9 mmol, 3.70 g). The reaction mixture was stirred at room temperature overnight. The solvent was removed under reduced pressure and the residue was purified by silica gel chromatography (MeOH:CHCl₃ 3:97) to afford tert-butyl (1-bromo-2-oxo-7,10,13-trioxa-3-azahexadecan-16-yl)carbamate S2 (34% yield, 1.86 g). TLC (MeOH:CHCl₃ 10:90), ¹H NMR (500 MHz, CDCl₃) δ 3.63-3.60 (m, 4H), 3.61-3.56 (m, 4H), 3.56-3.52 (m, 4H), 3.22 (d, J=6.0 Hz, 2H), 2.79 (t, J=6.7 Hz, 2H), 1.79-1.69 (m, 4H), 1.45 (s, 9H) ppm. ¹³C NMR (125 MHz, CDCl₃) δ 156.0, 78.7, 70.5, 70.5, 70.2, 70.1, 69.5, 69.4, 39.5, 38.4, 33.3, 29.5, 28.4 ppm. MS (ESI) [M+H]⁺ calcd. C₁₅H₃₂N₂O₅ 321.2, found 321.1.

### B). Synthesis of tert-butyl (1-bromo-2-oxo-7,10,13-trioxa-3-azahexadecan-16-yl) carbamate (S4)

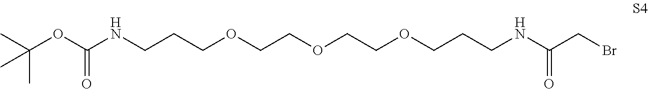 [SMILES: CC(C)(C)OC(=O)NCCCOCCOCCOCCCNC(=O)CBr]

Tert-butyl (3-(2-(2-(3-aminopropoxy)ethoxy)ethoxy)propyl)carbamate S2 (4.7 mmol, 1.5g) was dissolved in DCM (3 ml), in a 50 ml round bottom flask and K₂CO₃ (7 mmol, 1 g) in 3 ml of H₂O was added to it. Bromoacetyl bromide S3 (7 mmol, 1.4 g), dissolved in DCM (3 ml), was added drop wise to the mixture at 0-5° C. The reaction mixture was stirred for 12 h and the progress of the reaction was analyzed by using thin layer chromatography. Upon completion, the reaction mixture was extracted with DCM and the solution was concentrated under vacuum. The product was purified using silica gel column chromatography (MeOH:CHCl₃ 3:97) to afford tert-butyl (1-bromo-2-oxo-7,10,13-trioxa-3-azahexadecan-16-yl)carbamate S4 (75% yield, 1.5 g). TLC (MeOH:DCM 10:90), ¹H NMR (500 MHz, CDCl₃) δ 3.84 (s, 2H), 3.68-3.65 (m, 2H), 3.64-3.56 (m, 8H), 3.54 (t, J=6.0 Hz, 2H), 3.45-3.37 (m, 2H), 3.26-3.03 (m, 2H), 1.78-1.83 (m, 2H), 1.77-1.71 (m, 2H), 1.42 (s, 9H) ppm. ¹³C NMR (125 MHz, CDCl₃) δ 165.5, 156.0, 78.7, 70.5, 70.5, 70.2, 70.1, 69.5, 69.4, 39.5, 38.4, 33.3, 29.5, 28.4 ppm (One aliphatic carbon overlap). MS (ESI) [M+H]⁺ calcd. C₁₇H₃₃Br⁷⁹N₂O₆ 441.1, found 441.0 and calcd. C₁₇H₃₃Br⁸¹N₂O₆ 443.1, found 443.0.

### C). Synthesis of tert-butyl (1-(2-formylphenoxy)-2-oxo-7,10,13-trioxa-3-azahexadecan-16-yl)carbamate (S6)

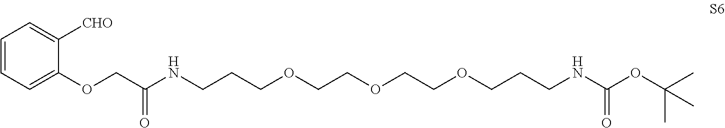 [SMILES: CC(C)(C)OC(=O)NCCCOCCOCCOCCCNC(=O)COc1ccccc1C=O]

In a 50 ml round bottom flask, 2-hydroxybenzaldehyde S5 (4.1 mmol, 500 mg) was dissolved in acetonitrile (8 ml). To this solution, K₂CO₃ (5.2 g, 37.7 mmol) and tert-butyl (1-bromo-2-oxo-7,10,13-trioxa-3-azahexadecan-16-yl)carbamate S4 (6 mmol, 828 mg) were added and the reaction mixture was allowed to reflux for 12 h. The reaction was monitored using thin layer chromatography. Upon completion, the reaction mixture was filtered to remove potassium carbonate. The solution was concentrated under vacuum and the product was purified using silica gel column chromatography (MeOH:DCM 5:95) to afford tert-butyl (1-(2-formylphenoxy)-2-oxo-7,10,13-trioxa-3-azahexadecan-16-yl)carbamate S6 (53% yield, 1.0 g). TLC (MeOH:DCM 10:90), ¹H NMR (500 MHz, CDCl₃) δ 10.25 (s, 1H), 7.79 (dd, J=7.6, 1.8 Hz, 1H), 7.66 (bs, 1H), 7.52-7.55 (m, 1H), 7.19-7.13 (m, 1H), 6.97-6.90 (m, 1H), 4.57 (s, 2H), 3.61-3.57 (m, 8H), 3.55-3.53 (m, 2H), 3.51-3.46 (m, 4H), 3.30-3.12 (m, 2H), 1.90-1.85 (m, 2H), 1.78-1.68 (m, 2H), 1.42 (s, 9H) ppm. ¹³C NMR (125 MHz, CDCl₃) δ 190.1, 167.5, 158.4, 156.2, 136.2, 133.0, 125.2, 122.1, 113.2, 79.0, 70.6, 70.6, 70.4, 70.3, 69.7, 69.5, 67.8, 38.7, 37.3, 29.8, 29.4, 28.6 ppm. MS (ESI) [M+H]⁺ calcd. C₂₄H₃₈N₂O₈ 483.2, found 483.1.

### D) Synthesis of N-(3-(2-(2-(3-aminopropoxy)ethoxy)ethoxy)propyl)-2-(2-formylphenoxy)acetamide 2c

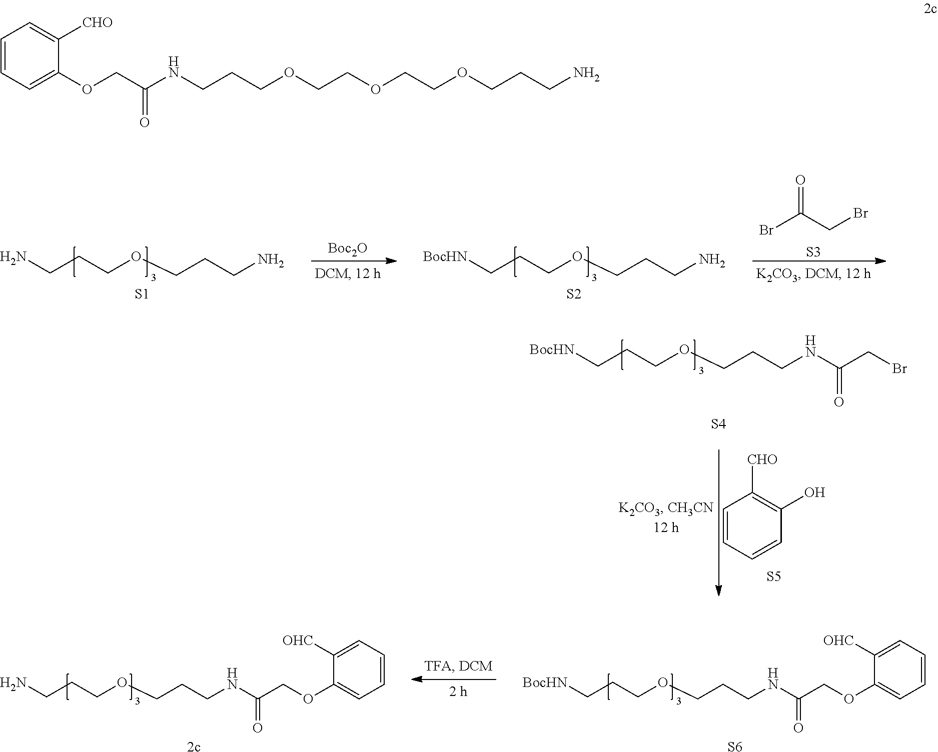

Procedure: In a 25 ml round bottom flask, tert-butyl (1-(2-formylphenoxy)-2-oxo-7,10,13-trioxa-3-azahexadecan-16-yl)carbamate S6 (500 mg, 1.3 mmol), was mixed with dichloromethane (3 ml). To this solution trifluoro acetic acid (1 ml) was added drop wise at 0-5° C. The reaction mixture was allowed to stir for 2 h. The reaction was monitored using thin layer chromatography and upon completion of the reaction, the solution was concentrated under vacuum to afford N-(3-(2-(2-(3-aminopropoxy)ethoxy)ethoxy)propyl)-2-(2-formylphenoxy)acetamide 2c (90% yield, 450 mg). Note: The oligomeric imine formation in deuterated solvent leads to complex NMR spectra. However, the LC and MS confirms the purity. HRMS (ESI) [M+H]⁺ calcd. C₁₉H₃₀N₂O₆ 383.2182, found 383.2192.

### E). Synthesis of N,N′-(((oxybis(ethane-2,1-diyl))bis(oxy))bis(propane-3,1-diyl))bis(2-bromoacetamide) (S7)

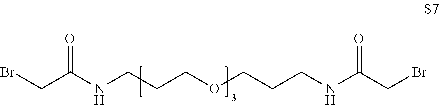

Procedure: 4,7,10-Trioxa-1,13-tridecanediamine S1 (9 mmol, 2 g) was dissolved in DCM (50 ml), in a 250 ml round bottom flask and K₂CO₃ (30 mmol, 4.1 g) in 20 ml of H₂O was added to it. Bromoacetyl bromide S3 (30 mmol, 6 g), dissolved in 20 ml of DCM was added drop wise to the mixture at 0-5° C. The reaction mixture was stirred for 12 h and the reaction progress was analyzed using thin layer chromatography. On completion of the reaction, reaction mixture was extracted with DCM. The collected organic fractions were dried over anhydrous sodium sulfate and filtered, the filtrate was concentrated under reduced pressure to afford N,N′-(((oxybis(ethane-2,1-diyl))bis(oxy))bis(propane-3,1-diyl))bis(2-bromoaceta mide) S7. Yield 72%; TLC (MeOH:DCM 10:90), ¹H NMR (500 MHz, CDCl₃) δ 3.85 (s, 4H), 3.68-3.64 (m, 4H), 3.64-3.56 (m, 8H), 3.41-3.39 (m, 4H), 1.81-1.78 (m, 4H) ppm. ¹³C NMR (125 MHz, CDCl₃) δ 165.4, 70.5, 70.3, 70.3, 38.9, 29.3, 28.5 ppm. HRMS (ESI) [M+H]⁺ calcd. C₁₄H₂₇Br₂N₂O₅ 463.0287, found 463.0267.

### F) Synthesis of N,N′-(((oxybis(ethane-2,1-diyl))bis(oxy))bis(propane-3,1-diyl))bis(2-(2-formylphenoxy)acetamide) (2b)

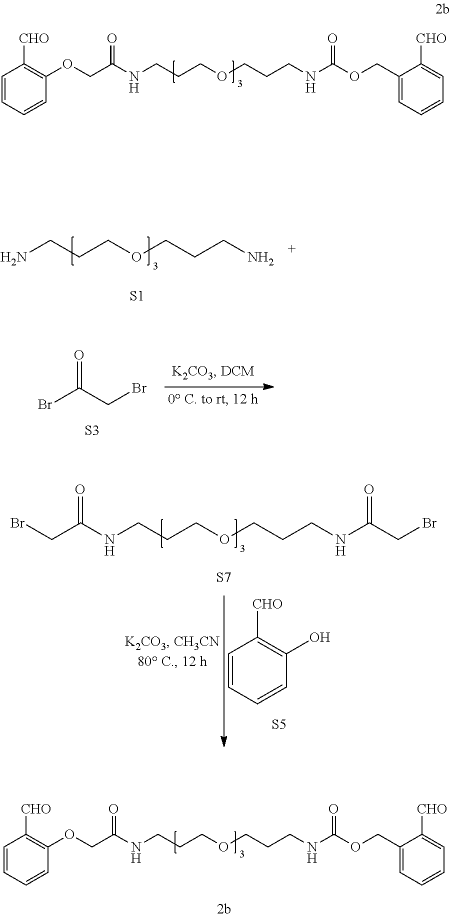

Procedure: In a 250 ml round bottom flask, 2-hydroxy benzaldehyde S5 (4.6 g, 37.7 mmol), N,N′-(((oxybis(ethane-2,1-diyl))bis(oxy))bis(propane-3,1-diyl))bis(2-bromoacetamide) S7 (3 g, 7 mmol) and K₂CO₃ (5.2 g, 37.7 mmol) were dissolved in acetonitrile (75 ml). The reaction mixture was allowed to reflux for 12 h. The reaction was monitored using thin layer chromatography and upon completion, the reaction mixture was filtered to remove potassium carbonate. The solution was concentrated under vacuum and the product was purified using flash column chromatography (MeOH:DCM 3:97) to afford N,N′-((oxybis(ethane-2,1-diyl))bis(oxy))bis(propane-3,1-diyl))bis(2-(2-formylphenoxy)acetamide) 2b. Yield 73%; MeOH:DCM 10:90, ¹H NMR (500 MHz, CDCl₃) δ 10.25 (s, 2H), 7.77-7.75 (m, 2H), 7.66 (bs, 2H), 7.59-7.53 (m, 2H), 7.12-7.09 (m, 2H), 6.92-6.88 (m, 2H), 4.55 (s, 4H), 3.56-3.51 (m, 12H), 3.48-3.42 (m, 4H), 1.87-1.80 (m, 4H) ppm. ¹³C NMR (125 MHz, CDCl₃) δ 190.0, 167.3, 158.4, 136.1, 132.6, 125.0, 121.9, 113.0, 70.4, 70.2, 69.3, 67.6, 37.0, 29.2 ppm. HRMS (ESI) [M+H]⁺ calcd. C₂₈H₃₆N₂O₉ 545.2499, found 545.2508.

### Example 2: Expression of SUMO1 Protein with N-Terminus Glycine

### Bacterial Transformation

E. coli strain [(DH5α for plasmid replication and BL21 (DE3) for protein expression] was used for transformation. The plasmid (1 μl) was added to the competent cells (50-100 μl) and was incubated on ice for 20 min. Subsequently, the heat shock was given at 42° C. for 40 seconds. The cells were kept on ice for 1 min, and 1 ml of LB was added to cells for recovery. The cells were incubated at 37° C., 180 rpm for 45 min. The recovered cells were plated on LB plates containing desired antibiotics. The plates were incubated at 37° C. for 12-16 hrs.

### Protein Purification

Primary culture was grown in LB overnight at 37° C. 1% of primary culture was sub-cultured into desired volume of LB media as secondary culture. At approximately 0.6-0.8 OD (600 nm), the secondary culture was induced with IPTG (200 μM) for 4 h at 30° C. for SUMO1. The induced culture was spun at 8000 rpm for 10 min to pellet down cells and the pellet was stored at −80° C.

For lysis, the cells were thawed on ice and resuspended in lysis buffer [20 mM Tris (pH 7.5), 150 mM NaCl, 1 mM EDTA, 5 mM β-ME]. Subsequently, 50 μg/ml lysozyme, 0.2% Triton X-100, 1X protease inhibitors 1 mM PMSF, Leupeptin, Pepstatin and Aprotinin mix, were added to facilitate cell lysis and protein stability. Lysate was incubated for 10-15 min in ice with constant shaking in between. This was followed by sonication (45% Amplitude, 10 sec ON 10 sec OFF cycle) till the suspension became clear. The supernatant was collected after spinning for 30 min at 11000 rpm, 4° C.

For protein binding and elution, the supernatant was transferred to column containing washed GSH beads. The protein bead binding was facilitated at 4° C. on the tumbler for 1 h. The beads were washed thrice with wash buffer [20 mM Tris (pH 7.5), 400 mM NaCl, 1 mM EDTA, 5 mM β-ME]. The protein was eluted in elution buffer [20 mM Tris (pH 8.0), 150 mM NaCl, 1 mM EDTA, 20 mM glutathione] and the concentration of eluted protein was determined using Bradford assay.

For clipping, protein-bound beads were washed thrice with prescission protease buffer [50 mM Tris (pH 7.5), 1 mM EDTA, 1 mM DTT, 150 mM NaCl, 0.1% triton]. The bead bound protein was quantified by Bradford method. In Prescission protease buffer, protein was clipped on beads using Prescission protease while maintaining the Prescission protease to total protein ratio 1:50. The clipping reaction proceeded at 4° C. for 18 h. The clipped proteins with N-terminus glycine were collected as supernatant, quantified and analyzed for their purity/stability on SDS-PAGE. The concentration of the sample was calculated using the spectrophotometric measurements.

The recombinantly expressed protein was further subjected to N-terminus glycine tagging with the N-terminus glycine capture reagent as in example 4 and reacted with resin for purification or reacted with the functionalized resin as in example 3 for purification.

### Example 3

### Procedure for N-Terminus Glycine Protein Purification

N-hydroxy succinimidyl sepharose beads 4 (400 μl, resin loading: 23 μmol/ml) were taken in a 5 ml fritted polypropylene chromatography column with end tip closures. Sodium bicarbonate buffer (0.1 M, pH 7.8, 3×1 ml) was used to wash the beads and were re-suspended (sodium bicarbonate buffer, 360 μl, 0.1 M, pH 7.8). To this solution, 2c (13.8 μM) in DMSO (40 μl) from a freshly prepared stock solution was added and vortexed at 25° C. The progress of the immobilization of the reagent on sepharose resin was monitored by UV-absorbance of the supernatant. Subsequently, the supernatant was removed and the beads were washed with aqueous buffer (0.1 M NaHCO₃/0.5 M NaCl pH 8.0, 3×1 ml; 0.1 M acetate/0.5 M NaCl pH 4.0, 3×1 ml) and H₂O (3×1 ml) to remove the adsorbed reagent from resin (store at 4° C.). The sepharose beads 5a were washed with the sodium bicarbonate buffer (0.1 M, pH 7.8, 3×1 ml) and re-suspended (sodium bicarbonate buffer, 375 μl, 0.1 M, pH 7.8). To this solution, native protein (20 nmol) dissolved in sodium bicarbonate buffer (25 μl, 0.1 M, pH 7.8) was added and vortexed at 25° C. Binding was ensured using UV-Vis analysis. The beads were washed thoroughly with aqueous buffer (0.1 M NaHCO₃/0.5 M NaCl pH 8.0, 3×1 ml), 1 N KCl (3×1 ml) and H₂O (3×1 ml) to remove any non-specifically bound protein from the resin. This was confirmed by analyzing the final wash fraction using LC-MS. For eluting out the bound protein, pyridoxal 5′-phosphate 12i (50 equiv.) in 0.1 M NaHCO₃ buffer, pH 7.8) was added to the resin and vortexed for 2 h at 25° C. The eluted protein was analysed by using ESI-MS.

### Example 4

### Labeling of Protein in Solution Phase

In a 1.5 ml Eppendorf tube, protein 1a (3 nmol) was mixed with sodium bicarbonate buffer (120 μl, 0.1 M, pH 7.8). To this solution, 2b (1500 nmol) in DMSO (30 μl) from a freshly prepared stock solution was added and vortexed at 25° C. The overall concentration of protein and 2b was 20 μM and 10 mM respectively. After 24-48 h, the reaction mixture was diluted with acetonitrile:water (10:90, 3000 μl). Unreacted 2-(2-formylphenoxy)acetic acid and salts were removed by using Amicon® Ultra-0.5 mL 3-kDa or 10-kDa MWCO centrifugal filters spin concentrator. The protein mixture was further washed with Millipore Grade I water (5×0.4 ml). The sample was analyzed by ESI-MS. The aqueous sample was concentrated by lyophilization before subjecting it to digestion, peptide mapping, and sequencing by MS-MS.

### Example 5

The hydrazide functionalized resin (200 μl, resin loading: 16 μmol/ml) were taken in a 5 ml fritted polypropylene chromatography column. After wash with phosphate buffer (0.1 M, pH 7.0, 5×1 ml), the resin was re-suspended in phosphate buffer (100 μl, 0.1 M, pH 7.0). The protein mixture from example 4 containing 2b treated 1a (250 μM) in phosphate buffer (150 μl, 0.1 M, pH 7.0) and aniline (100 mM) in phosphate buffer (100 μl, 0.1 M, pH 7.0) were added to the resin followed by end-to-end rotation (30 rpm, rotary mixer) at 25° C. The progress of the immobilization of the labeled protein on hydrazide resin was monitored by UV-absorbance of the supernatant. After 8-10 h, the supernatant was collected and the beads were washed with phosphate buffer (0.3 M, pH 7.3, 4×1 ml) and KCl (1 M, 3×1 ml) to remove the adsorbed protein from resin. The resin was further washed with the phosphate buffer (0.3 M, pH 7.0, 4×1 ml) and re-suspended (phosphate buffer, 200 μl, 0.3 M, pH 7.0). To release the labeled protein from its immobilized derivative, aniline (100 mM) in phosphate buffer (100 μl, 0.3 M, pH 7.0) and coumarin or fluoro or biotin derivatives (only one at a time) of O-hydroxylamine (50 μl, 150 mM in DMSO) were added followed by vortex at 25° C. for 6-8 h. The supernatant was collected while the salts, aniline and O-hydroxylamine were removed using the spin concentrator (3 kDa MWCO). The purity of the labeled protein was confirmed by ESI-MS. Further analysis was performed using NMR or SDS-PAGE or fluorescence spectroscopy. After all the analysis, the probe was removed through C—C bond dissociation using pyridoxal 5′-phosphate 12i (50 equiv.) in 0.1 M NaHCO₃ buffer, pH 7.8) by vortexing it for 2 h at 25° C. The final POI was analysed by using ESI-MS.

### Advantages of the Invention

The method provides N-terminus Glycine specific labelling of proteins.

The method provides metal-free covalent affinity purification of proteins.

The method of the invention results is efficient selective capture of the protein of interest (POI) with N-terminus Glycine tagged protein while leaving the other proteins in solution.

The method of the invention is effective for C—C bond formation under mild conditions.

The method of the invention is effective for C—C bond dissociation under mild conditions.

The cost of operation is reduced because of the recovery and recycling of the functionalized sepharose resin.

The method of the invention facilitates the separation and isolation of N-terminus Glycine tagged proteins from a mixture of proteins with or without probes.

The method of the invention is advantageous for purification of the N-terminus Glycine tagged protein from a cell lysate.

### References

Adusumalli, S. R.; Rawale, D. G.; Singh, U.; Tripathi, P.; Paul, R.; Kalra, N.; Mishra, R. K.; Shukla, S.; Rai, V. J. Am. Chem. Soc. 2018, 140, 15114-15123. (Single-site labeling of native proteins enabled by a chemoselective and site-selective chemical technology)

Usera; Aimee et al 2015, US Patent 20150017192. (Site-specific chemoenzymatic protein modifications)

Hober; Sophia et al 2013, US Patent 20130184442. Method for labeling of compounds)

Schultz et al 2012, US Patent 20120202243. (In vivo incorporation of unnatural amino acids)

Schultz et al 2015, US Patent 20150018523. (Unnatural reactive amino acid genetic code additions)

Davis et al 2011, US Patent 20110059501. (Protein glycosylation)

Noren, C. J.; Anthony-Cahill, S. J.; Griffith, M. C.; Schultz, P. G. Science, 1989, 244, 182-188. (A General Method for Site-Specific Incorporation of Unnatural Amino Acids into Proteins)

Cornish, V. W.; Benson, D. R.; Altenbach, C. A. Hideg, K.; Hubbell, W. L.; Schultz, P. G. Proc. Natl. Acad. Sci. (USA), 1994, 91, 2910-2914. (Site Specific Incorporation of Biophysical Probes into Proteins)

Kim, C.; Axup, J.; Schultz, P. G. Curr. Opin. Chem. Biol. 2013, 17, 412-419. Protein conjugation with genetically encoded unnatural amino acids)

Xiao, H.; Chatterjee, A.; Choi, S.; Bajjuri, K. M.; Sinha, S. C.; Schultz, P. G.; Angew. Chem. Int. Ed. 2013, 52, 14080-14083. (Genetic incorporation of multiple unnatural amino acids into proteins into mammalian cells)

Chalker, J. M.; Bernardes, G. J. L.; Davis, B. G. Acc. Chem. Res., 2011, 44, 730-741. (A “Tag-and-Modify” Approach to Site-Selective Protein Modification)

Krueger, A. T.; Imperiali, B. ChemBioChem 2013, 14, 788-799. (Fluorescent Amino Acids: Modular Building Blocks for the Assembly of New Tools for Chemical Biology)

Smith, E. L.; Giddens, J. P.; Iavarone, A. T.; Godula, K.; Wang, L. X.; Bertozzi, C. R. Bioconjug. Chem. 2014, 25,788-795. (Chemoenzymatic Fc Glycosylation via Engineered Aldehyde Tags.

1. A method for metal free purification of protein from a protein mixture or cell lysate comprising the steps of:
preparing a N-terminus glycine capture reagent;
preparing a functionalized resin with N-terminus glycine capture reagent;
reacting the N-terminus glycine containing proteins from the protein mixture or cell lysate with the functionalized resin to form a C—C bond association and a stable amino alcohol;
immobilising the N-terminus glycine containing proteins in an ordered pattern from the protein mixture or cell lysate on the functionalized resin;
separating the N-terminus glycine proteins from the functionalised resin under aqueous physiological conditions by C—C bond dissociation with additive, wherein the additive enables the resonance-assisted electron density (RED) polarization to facilitate C—C bond dissociation; and recovery and recycling of the functionalized resin without substantial loss of activity.

2. The method as claimed in claim 1, wherein the N-terminus glycine capture reagent is selected from the compounds of formula 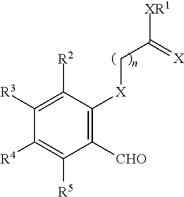
where, X is a heteroatom (O, N, S), n is 1-6 and R¹-R⁵ are independently selected H; alkyl; lower alkyl; cycloalkyl; aryl; heteroaryl; alkenyl; heterocycle; halides; nitro; —C(O)OR* wherein R* is selected from H, alkyl; cycloalkyl and aryl; —C(O)NR**R***, wherein R** and R*** are independently selected from H, alkyl; cycloalkyl and aryl; —CH₂C(O)Rₐ, wherein Rₐ is selected from —OH, lower alkyl, cycloalkyl; aryl, -lower alkyl-aryl, -cycloalkyl-aryl; or —NR_{b}R_{c}, where R_{b} and R_{c} are independently selected from H, lower alkyl, cycloalkyl; aryl or -lower alkyl-aryl; —C(O)R_{d}, wherein R_{d} is selected from lower alkyl, cycloalkyl; aryl or -lower alkyl-aryl; or -lower alkyl-ORₑ, wherein Rₑ is a suitable protecting group or OH group. R¹ group can also be selected from an amino acid, small peptide, large peptide, a protein, an antibody, their unnatural derivatives or other biomolecules bearing —CH₂NH₂ group. Small peptide is a 2-mer to 10-mer peptide and large peptide is 11-mer to 30-mer peptide. All the Rⁿ groups are optionally substituted at one or more substitutable positions with one or more suitable substituents;
the “suitable substituent” includes independently H; hydroxyl; cyano; alkyl, such as lower alkyl, such as methyl, ethyl, propyl, n-butyl, t-butyl, hexyl and the like; alkoxy, such as lower alkoxy such as methoxy, ethoxy, and the like; aryloxy, such as phenoxy and the like; vinyl; alkenyl, such as hexenyl and the like; alkynyl; formyl; haloalkyl, such as lower haloalkyl which includes CF₃, CCl₃ and the like; halide; aryl, such as phenyl and napthyl; heteroaryl, such as thienyl and furanyl and the like; amide such as C(O)NR**R***, where R** and R*** are independently selected from lower alkyl, aryl or benzyl, and the like; acyl, such as C(O)—C₆H₅, and the like; ester such as —C(O)OCH₃ the like; ethers and thioethers, such as O—Bn and the like; thioalkoxy;
phosphino; and —NR_{b}R_{c}, where R_{b} and R_{c} are independently selected from lower alkyl, aryl or benzyl, and the like. The term “lower alkyl” as used herein either alone or in combination with another substituent means acyclic, straight or branched chain alkyl substituent containing from one to six carbons and includes for example, methyl, ethyl, 1-methylethyl, 1-methylpropyl, 2-methylpropyl, and the like. A similar use of the term is to be understood for “lower alkoxy”, “lower thioalkyl”, “lower alkenyl” and the like in respect of the number of carbon atoms. For example, “lower alkoxy” as used herein includes methoxy, ethoxy, t-butoxy;
the term “alkyl” encompasses lower alkyl, and also includes alkyl groups having more than six carbon atoms, such as, for example, acyclic, straight or branched chain alkyl substituents having seven to ten carbon atoms;
the term “aryl” as used herein, either alone or in combination with another substituent, means an aromatic monocyclic system or an aromatic polycyclic system. For example, the term “aryl” includes a phenyl or a napthyl ring, and may also include larger aromatic polycyclic systems, such as fluorescent (eg. anthracene) or radioactive labels and their derivatives;
the term “heteroaryl” as used herein, either alone or in combination with another substituent means a 5, 6, or 7-membered unsaturated heterocycle containing from one to 4 heteroatoms selected from nitrogen, oxygen, and sulphur and which form an aromatic system. The term “heteroaryl” also includes a polycyclic aromatic system comprising a 5, 6, or 7-membered unsaturated heterocycle containing from one to 4 heteroatoms selected from nitrogen, oxygen, and sulphur;
the term “cycloalkyl” as used herein, either alone or in combination with another substituent, means a cycloalkyl substituent that includes for example, but is not limited to, cyclopropyl, cyclobutyl, cyclopentyl, cyclohexyl and cycloheptyl. The term also involves “cycloalkyl-alkyl-” that means an alkyl radical to which a cycloalkyl radical is directly linked; and includes, but is not limited to, cyclopropylmethyl, cyclobutylmethyl, cyclopentylmethyl, 1-cyclopentylethyl, 2-cyclopentylethyl, cyclohexylmethyl, 1-cyclohexylethyl and 2-cyclohexylethyl. A similar use of the “alkyl” or “lower alkyl” terms is to be understood for aryl-alkyl-, aryl-lower alkyl- (eg. benzyl), -lower alkyl-alkenyl (eg. allyl), heteroaryl-alkyl-, and the like as used herein. For example, the term “aryl-alkyl-” means an alkyl radical, to which an aryl is bonded. Examples of aryl-alkyl- include, but are not limited to, benzyl (phenylmethyl), 1-phenylethyl, 2-phenylethyl and phenylpropyl. As used herein, the term “heterocycle”, either alone or in combination with another radical, means a monovalent radical derived by removal of a hydrogen from a three- to seven-membered saturated or unsaturated (including aromatic) heterocycle containing from one to four heteroatoms selected from nitrogen, oxygen and sulfur. Examples of such heterocycles include, but are not limited to, pyrrolidine, tetra-hydrofuran, thiazolidine, pyrrole, thiophene, hydantoin, diazepine, imidazole, isoxazole, thiazole, tetrazole, piperidine, piperazine, homopiperidine, homo-piperazine, 1,4-dioxane, 4-morpholine, 4-thiomorpholine, pyridine, pyridine-N-oxide or pyrimidine, and the like;
the term “alkenyl”, as used herein, either alone or in combination with another radical, is intended to mean an unsaturated, acyclic straight chain radical containing two or more carbon atoms, at least two of which are bonded to each other by a double bond. Examples of such radicals include, but are not limited to, ethenyl (vinyl), 1-propenyl, 2-propenyl, and 1-butenyl. The term “alkynyl”, as used herein is intended to mean an unsaturated, acyclic straight chain radical containing two or more carbon atoms, at least two of which are bonded to each other by a triple bond. Examples of such radicals include, but are not limited to, ethynyl, 1-propynyl, 2-propynyl, and 1-butynyl;
the term “alkoxy” as used herein, either alone or in combination with another radical, means the radical —O—(C₁₋ₓ) alkyl wherein alkyl is as defined above containing 1 or more carbon atoms, and includes for example methoxy, ethoxy, propoxy, 1-methylethoxy, butoxy and 1,1-dimethylethoxy. Where x is 1 to 6, the term “lower alkoxy” applies, as noted above, whereas the term “alkoxy” encompasses “lower alkoxy” as well as alkoxy groups where x is greater than 6 (for example, x=7 to 10). The term “aryloxy” as used herein alone or in combination with another radical means —O-aryl, wherein aryl is defined as noted above.

3. The method as claimed in claim 2, wherein the N-terminus glycine capture reagent is preferably N-(3-(2-(2-(3-aminopropoxy)ethoxy)ethoxy)propyl)-2-(2-formylphenoxy)acetamide.

4. The method as claimed in claim 1, wherein the aqueous physiological condition is at pH of 7±1.

5. The method as claimed in claim 1, wherein the resin for functionalisation is selected from one of NHS Sepharose, NHS Agarose, and the like.

6. The method as claimed in claim 1, wherein the additive for C—C bond dissociation is selected from one of 4-dimethyl amino pyridine (DMAP), 1,8-Diazabicyclo[5.4.0]undec-7-ene (DBU), 1,5-Diazabicyclo[4.3.0]non-5-ene (DBN), 1,4-Diazabicyclo[2.2.2]octane (DABCO), Imidazole, N-methyl Imidazole, triethyl amine, pyridoxal-5-phosphate (PLP), or other RED polarization promoting additives.

7. The method as claimed in claim 6, wherein the additive for C—C bond dissociation is preferably pyridoxal-5-phosphate.

8. The method as claimed in claim 1, wherein the recovered functionalized resin is used for 5-7 purification cycles.

9. A method for metal free purification of protein from a protein mixture or cell lysate comprising the steps of:
preparing an N-terminus glycine capture reagent as claimed in claim 2;
reacting the N-terminus glycine capture reagent with N-terminus glycine containing proteins in an aqueous phase from the protein mixture or cell lysate to form N-terminus glycine tagged protein;
reacting N-terminus glycine tagged proteins with the resin or a probe to form a C—C bond association and a stable amino alcohol;
separating the N-terminus glycine proteins from the resin or a probe under aqueous physiological conditions by C—C bond dissociation with an additive, wherein the additive enables the resonance-assisted electron density (RED) polarization to facilitate C—C bond dissociation; and optionally recovering and recycling as functionalised resin or a probe without substantial loss of activity.

10. The method as claimed in claim 9, wherein the N-terminus glycine capture reagent is preferably N,N′-(((oxybis(ethane-2,1-diyl))bis(oxy))bis(propane-3,1-diyl))bis(2-(2-formylphenoxy)acetamide).

11. The method as claimed in claim 9, wherein the resin is selected from one of NHS Sepharose, NHS Agarose, and the like.

12. The method as claimed in claim 9, wherein the probe is selected from one of biotin, fluorophore, biophysical probe, and the like.

13. The method as claimed in claim 9, wherein the additive for C—C bond dissociation is selected from one of 4-dimethyl amino pyridine (DMAP), 1,8-Diazabicyclo[5.4.0]undec-7-ene (DBU), 1,5-Diazabicyclo[4.3.0]non-5-ene (DBN), 1,4-Diazabicyclo[2.2.2]octane (DABCO), Imidazole, N-methyl Imidazole, triethyl amine, pyridoxal-5-phosphate (PLP), or other RED polarization promoting additives.

14. The method as claimed in claim 7, wherein the additive for C—C bond dissociation is preferably pyridoxal-5-phosphate.

15. The method as claimed in claim 6, wherein the recovered functionalized resin is used for 5-7 purification cycles.
